(19) United States
(12) Patent Application Publication
(10) Pub. No.: US 2026/0275209 A1
(43) Pub. Date: Sep. 17, 2026

(54) METHOD FOR CATALYTIC REFORMING WITH UPGRADING OF NON-AROMATIC EFFLUENTS

(71) Applicant: IFP Energies nouvelles, Rueil-Malmaison (FR)

(72) Inventors: Marie DEHLINGER, Rueil-Malmaison Cedex (FR); Celine BERTINO-GHERA, Rueil-Malmaison Cedex (FR); Faustine CLAIRE, Rueil-Malmaison (FR)

(73) Assignee: IFP Energies nouvelles, Rueil-Malmaison (FR)

(21) Appl. No.: 19/472,743

(22) PCT Filed: Mar. 29, 2024
(86) PCT No.: PCT/EP2024/058786

§ 371 Date: Oct. 6, 2025

(30) Foreign Application Priority Data
Apr. 7, 2023 (FR) FR2303523

(51) Int. Cl. C10G 67/14

(52) U.S. Cl. CPC C10G 67/14; C10G 2300/1044; C10G 2300/1096; C10G 2400/30

(57) ABSTRACT

According to a first aspect, the present invention relates to a process and apparatus for producing aromatic compounds. in which the raffinate (16) from an aromatics extraction unit (14) is sent to a dedicated catalytic reforming unit (17) to produce a reformate exit stream (18) that is successively treated by: a recontacting section (4) to separate hydrogen (5); a stabilization section (7) to separate C1-C2 hydrocarbon compounds (8) and C3-C4 or C3-C5 hydrocarbon compounds (9); and a reformate separation column (11) to separate a C6-C8 fraction (12) and a C8+ fraction (13), the C6-C8 fraction (12) being sent to the aromatics extraction unit (14). The present invention also relates to a process for modifying an existing apparatus for producing aromatic compounds, comprising installing the process according to the first aspect in the existing apparatus.

### TECHNICAL FIELD

The present invention relates to the field of processes for producing aromatic compounds and more particularly aromatic compounds of benzene, toluene and xylene type by catalytic reforming of a hydrocarbon feedstock of naphtha type.

### PRIOR ART

Generally, the object of a catalytic reforming unit is to convert naphthenic and paraffinic (n-paraffin and isoparaffin) compounds into aromatic compounds. The main reactions involved are the dehydrogenation of naphthenes and the dehydrocyclization of paraffins to give aromatics, the isomerization of paraffins and the isomerization of naphthenes. Other “side” reactions may also take place, such as the hydrocracking and hydrogenolysis of paraffins and naphthenes, the hydrodealkylation of alkylaromatics, giving rise to light compounds and lighter aromatics, and also coke formation at the surface of the catalysts.

The feedstocks typically sent to a catalytic reforming unit are rich in paraffinic and naphthenic compounds and relatively poor in aromatic compounds. They are generally naphthas from the distillation of crude oil or of natural gas condensates. Other feedstocks may also be available, containing variable contents of aromatics, namely heavy naphthas from catalytic cracking, coking or hydrocracking, or petrols from steam cracking.

For use in petrochemistry, the performance qualities sought are the yield of aromatics and also the distribution of the aromatics produced. Aromatic compounds are generally treated in an aromatic complex in order to favour the production of one or more aromatic compounds, most commonly xylenes (especially para-xylene) and benzene. An aromatic complex is an aromatics production unit comprising for example separation sections (for example distillation, adsorption/desorption, crystallization) and optionally transformation sections (for example isomerization, transalkylation) for the production of aromatic compounds, especially para-xylene. Toluene and the heavier aromatics can be upgraded so as to form petrol bases, or through the production of a mixture of xylenes. The production of C6-C7 aromatic compounds makes it possible in particular to improve the octane number of the petrol and/or makes it possible to increase the supply of benzene, toluene and xylenes. In order to maximize the production of C6-C7 aromatic compounds by catalytic reforming, the presence of a specific catalytic system generally comprising a catalytically active metal (generally platinum) and a non-acid zeolite is used.

A conventional catalytic reforming scheme for the production of aromatics, without upgrading the raffinate from the extraction of aromatics, from a feedstock of naphtha type comprises the following steps:
sending a naphtha stream to a reforming unit, generating an exit stream;
sending the exit stream to a reformate separation column, thereby creating a top stream and a bottom stream;
sending the top stream to a unit for the purification of aromatic compounds, thereby creating a purified aromatics stream comprising C6 and C7 aromatic compounds, and a raffinate stream.

There are many other known processes that aim to increase production of C6-C7 aromatic compounds from the conventional process. For example, patent application US2012/0277505 describes the conventional process mentioned above in which the raffinate stream is recycled to the reforming unit.

Patent application US2012/0277505 also describes a process for improving the production of benzene and toluene from a feedstock of naphtha type comprising the following steps:
sending a naphtha stream to a fractionation unit, generating a first stream comprising C7 and lighter hydrocarbons and a second stream comprising heavier hydrocarbons;
sending the first stream to a first reforming unit, generating a first exit stream;
sending the second stream to a second reforming unit, at a temperature greater than the temperature applied in the first reforming unit, generating a second exit stream;
sending the first exit stream and the second exit stream to a reformate separation column, thereby creating a top stream and a bottom stream;
sending the top stream to a unit for the purification of aromatic compounds, thereby creating a purified aromatics stream comprising C6 and C7 aromatic compounds, and a raffinate stream;
recycling the raffinate stream to the first reforming unit.

Such processes result in increased production of aromatic hydrocarbons, and of benzene and of toluene in particular, from a naphtha feed stream. More particularly, the step of recycling the raffinate and the repositioning of the aromatics extraction unit in relation to the catalytic reforming units situated in parallel makes it possible to generate an increase of 25% in benzene yields and an increase of approximately 10% in toluene yields.

However, although these processes are aimed at increasing the production of C6-C7 aromatic compounds, the production of xylenes can be improved. In addition, the improvements proposed are often poorly suited to the modification of existing units, the existing aromatic complex having insufficient capacity for treating new aromatic compounds.

### SUMMARY OF THE INVENTION

In the context described above, a first object of the present description is to overcome the problems of the prior art and to provide a catalytic reforming process and apparatus that make it possible to increase the production of benzene and toluene. A second object of the present description is to provide a catalytic reforming process and apparatus that can be implemented in an existing complex without modifying the existing installations, especially with a view to increasing the production of benzene and xylenes.

According to a first aspect, the abovementioned objects, and also other advantages, are obtained by a process for producing aromatic compounds, such as aromatics having 6-8 carbon atoms (A6-A8), and xylenes in particular, from a hydrocarbon compound feedstock, comprising the following steps:
sending a first C6-C8 hydrocarbon compound feedstock to an aromatics extraction unit so as to produce an extract comprising aromatic compounds having 6 to 8 carbon atoms and a raffinate comprising aliphatic compounds having 6 to 8 carbon atoms;
sending the raffinate to a first dedicated catalytic reforming unit so as to produce a first reformate exit stream;
sending the first reformate exit stream to a first recontacting section so as to separate a first hydrogen-rich gaseous exit stream and a first liquid hydrocarbon exit stream;
sending the first liquid hydrocarbon exit stream to a first stabilization section so as to separate a first gaseous hydrocarbon compound exit stream comprising C1-C2 hydrocarbon compounds, a first liquid stream comprising C3-C4 or C3-C5 hydrocarbon compounds, and a first liquid fraction comprising hydrocarbon compounds having at least 4 or 5 carbon atoms and preferably at least 5 carbon atoms;
sending the first liquid fraction to a first reformate separation column so as to separate a first C6-C8 fraction comprising C6-C8 hydrocarbon compounds and a first C8+ fraction comprising hydrocarbon compounds having at least 8 carbon atoms;
sending the first C6-C8 fraction at least in part to the aromatics extraction unit.

According to one or more embodiments, the process comprises sending the extract and the first C8+ fraction to an aromatics production unit (aromatic complex).

According to one or more embodiments, the raffinate is sent to a first dedicated hydrotreating unit so as to produce a first hydrotreated exit stream sent to the first catalytic reforming unit.

According to one or more embodiments, the first C6-C8 fraction comprises C5 hydrocarbon compounds, and/or a first top fraction comprising C5 hydrocarbon compounds is separated in the first reformate separation column.

According to one or more embodiments, the first C6-C8 hydrocarbon compound feedstock comprises at least 50% by weight, preferably at least 60% by weight, very preferably at least 70% by weight, of aromatic compounds.

According to one or more embodiments, the first catalytic reforming unit comprises at least one reforming reactor operated according to at least one of the following operating conditions:
a temperature of between 400° C. and 600° C., preferably between 470° C. and 570° C.;
a pressure of between 0.1 MPa and 3 MPa, preferably between 0.3 MPa and 2.5 MPa;
a molar ratio between hydrogen and hydrocarbon compounds of between 0.8 and 8 mole/mole; and
a mass flow rate of treated stream per unit mass of catalyst and per hour of between 0.1 h⁻¹ and 10 h⁻¹, preferably between 0.5 h⁻¹ and 6 h⁻¹;
optional catalyst comprising: an active phase comprising at least one metal selected from platinum, zinc or molybdenum, preferably platinum; a support comprising a zeolite, preferably a zeolite selected from a zeolite L, a zeolite X, a zeolite Y or a zeolite ZSM-5; and optionally a binder selected from aluminosilicate, alumina, silica, clays or silicon carbides, taken alone or in combination.

According to one or more embodiments, the process comprises the following steps:
sending a second hydrocarbon feedstock to a second catalytic reforming unit so as to produce a second reformate exit stream;
sending the second reformate exit stream to a second recontacting section so as to produce a second hydrogen-rich gaseous exit stream and a second liquid hydrocarbon exit stream;
sending the second liquid hydrocarbon exit stream to a second stabilization section so as to separate a second gaseous hydrocarbon compound exit stream comprising C1-C2 hydrocarbon compounds, a second liquid stream comprising C3-C4 or C3-C5 hydrocarbon compounds, and a second liquid fraction comprising hydrocarbon compounds having at least 4 or 5 carbon atoms and preferably at least 5 carbon atoms;
sending the second liquid fraction to a second reformate separation column so as to separate a second C6-C8 fraction and a second C8+ fraction comprising hydrocarbon compounds having at least 8 carbon atoms;
sending the second C6-C8 fraction to the aromatics extraction unit as said first C6-C8 hydrocarbon compound feedstock.

According to one or more embodiments, the process comprises the following steps:
sending a second hydrocarbon feedstock to a second catalytic reforming unit so as to produce a second reformate exit stream;
sending the second reformate exit stream to a second recontacting section so as to produce a second hydrogen-rich gaseous exit stream and a second liquid hydrocarbon exit stream;
sending the second liquid hydrocarbon exit stream to a second stabilization section so as to separate a second gaseous hydrocarbon compound exit stream comprising C1-C2 hydrocarbon compounds, a second liquid stream comprising C3-C4 or C3-C5 hydrocarbon compounds, and a second liquid fraction comprising hydrocarbon compounds having at least 4 or 5 carbon atoms and preferably at least 5 carbon atoms;
sending the second liquid fraction to the first reformate separation column.

According to one or more embodiments, the process comprises the following steps:
sending a second hydrocarbon feedstock to a second catalytic reforming unit so as to produce a second reformate exit stream;
sending the second reformate exit stream to a second recontacting section so as to produce a second hydrogen-rich gaseous exit stream and a second liquid hydrocarbon exit stream;
sending the second liquid hydrocarbon exit stream to the first stabilization section.

According to one or more embodiments, the process comprises the following steps:
sending a second hydrocarbon feedstock to a second catalytic reforming unit so as to produce a second reformate exit stream;
sending the second reformate exit stream at least in part to the first recontacting section.

According to one or more embodiments, the process comprises sending the second C8+ fraction at least in part to an aromatics production unit.

According to one or more embodiments, the second hydrocarbon feedstock is sent to a second dedicated hydrotreating unit so as to produce a second hydrotreated exit stream sent to the second catalytic reforming unit.

According to one or more embodiments, the second hydrocarbon feedstock comprises naphtha.

According to a second aspect, the abovementioned objects, and also other advantages, are obtained by a process for modifying an apparatus for producing aromatic compounds, such as aromatics having 6-8 carbon atoms (A6-A8), and xylenes in particular, from a hydrocarbon compound feedstock,
the apparatus for producing aromatic compounds comprising:
   a second catalytic reforming unit suitable for treating a second hydrocarbon feedstock and producing a second reformate exit stream;
   a second recontacting section suitable for treating the second reformate exit stream and producing a second hydrogen-rich gaseous exit stream and a second liquid hydrocarbon exit stream;
   a second stabilization section suitable for treating the second liquid hydrocarbon exit stream and separating a second gaseous hydrocarbon compound exit stream comprising C1-C2 hydrocarbon compounds, a second liquid stream comprising C3-C4 or C3-C5 hydrocarbon compounds, and a second liquid fraction comprising hydrocarbon compounds having at least 4 or 5 carbon atoms and preferably at least 5 carbon atoms;
   a second reformate separation column suitable for treating the second liquid fraction and separating a second C6-C8 fraction and a second C8+ fraction comprising hydrocarbon compounds having at least 8 carbon atoms;
   an aromatics extraction unit suitable for treating the second C6-C8 fraction and producing an extract comprising aromatic compounds having 6 to 8 carbon atoms and a raffinate comprising aliphatic compounds having 6 to 8 carbon atoms, the process comprising the following steps:
   installing in the apparatus for producing aromatic compounds a first dedicated catalytic reforming unit suitable for treating the raffinate, producing a first reformate exit stream, and sending the first reformate exit stream to the second recontacting section or to a first dedicated recontacting section;
   optionally, installing in the apparatus for producing aromatic compounds the first dedicated recontacting section suitable for treating the first reformate exit stream, separating a first hydrogen-rich gaseous exit stream and a first liquid hydrocarbon exit stream, and sending the first liquid hydrocarbon exit stream to the second stabilization section or to a first dedicated stabilization section;
   optionally, installing in the apparatus for producing aromatic compounds the first dedicated stabilization section suitable for treating the first liquid hydrocarbon exit stream, separating a first gaseous hydrocarbon compound exit stream comprising C1-C2 hydrocarbon compounds, a first liquid stream comprising C3-C4 or C3-C5 hydrocarbon compounds, and a first liquid fraction comprising hydrocarbon compounds having at least 4 or 5 carbon atoms and preferably at least 5 carbon atoms, and sending the first liquid fraction to the second reformate separation column or to a first dedicated reformate separation column.
   optionally, installing in the apparatus for producing aromatic compounds the first dedicated reformate separation column suitable for treating the first liquid fraction, separating a first C6-C8 fraction comprising C6-C8 hydrocarbon compounds and a first C8+ fraction comprising hydrocarbon compounds having at least 8 carbon atoms, and sending the first C6-C8 fraction at least in part to the aromatics extraction unit.

According to a third aspect, the abovementioned objects, and also other advantages, are obtained by an apparatus for producing aromatic compounds, such as aromatics having 6-8 carbon atoms (A6-A8), and xylenes in particular, from a hydrocarbon compound feedstock, the apparatus comprising:
an aromatics extraction unit suitable for treating a first C6-C8 hydrocarbon compound feedstock so as to produce an extract comprising aromatic compounds having 6 to 8 carbon atoms and a raffinate comprising aliphatic compounds having 6 to 8 carbon atoms;
a first dedicated catalytic reforming unit suitable for treating the raffinate and producing a first reformate exit stream;
a first recontacting section suitable for treating the first reformate exit stream and separating a first hydrogen-rich gaseous exit stream and a first liquid hydrocarbon exit stream;
a first stabilization section suitable for treating the first liquid hydrocarbon exit stream and separating a first gaseous hydrocarbon compound exit stream comprising C1-C2 hydrocarbon compounds, a first liquid stream comprising C3-C4 or C3-C5 hydrocarbon compounds, and a first liquid fraction comprising hydrocarbon compounds having at least 4 or 5 carbon atoms and preferably at least 5 carbon atoms;
a first reformate separation column suitable for treating the first liquid fraction, separating a first C6-C8 fraction comprising C6-C8 hydrocarbon compounds and a first C8+ fraction comprising hydrocarbon compounds having at least 8 carbon atoms, and sending the first C6-C8 fraction at least in part to the aromatics extraction unit.

Embodiments of the invention according to the abovementioned aspects and also other features and advantages will become apparent on reading the description that follows, given solely by way of illustration and in a non-limiting manner, and with reference to the drawings hereinbelow.

### LIST OF FIGURES

FIG. 1 is a simplified schematic representation of the process for producing aromatic compounds according to the invention.

FIG. 2 is a simplified schematic representation of the process for producing aromatic compounds according to FIG. 1 in parallel with conventional reforming.

FIG. 3 is a simplified schematic representation of the process for producing aromatic compounds according to FIG. 2 with a shared reformate separation column.

FIG. 4 is a simplified schematic representation of the process for producing aromatic compounds according to FIG. 3 with a shared stabilization section.

FIG. 5 is a simplified schematic representation of the process for producing aromatic compounds according to FIG. 4 with a shared recontacting section.

### DESCRIPTION OF THE EMBODIMENTS

The present invention relates to a process for producing aromatic compounds, such as aromatics having 6-8 carbon atoms (A6-A8), and xylenes in particular, from a hydrocarbon compound feedstock.

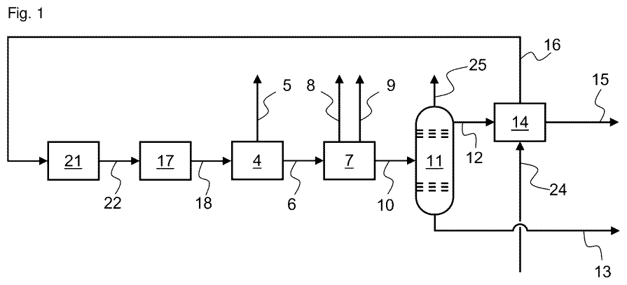

With reference to FIG. 1, according to the invention, the process comprises the following steps:
sending a first C6-C8 hydrocarbon compound feedstock 24 to an aromatics extraction unit 14 so as to form an extract 15 comprising (predominantly, preferably essentially) aromatic compounds having 6 to 8 carbon atoms and a raffinate 16 comprising (predominantly, preferably essentially) aliphatic compounds having 6 to 8 carbon atoms;
sending the raffinate 16 to a first dedicated catalytic reforming unit 17 so as to obtain a first reformate exit stream 18;
sending the first reformate exit stream 18 to a first recontacting section 4 (optionally dedicated) so as to obtain a first hydrogen-rich gaseous exit stream 5 (more hydrogen-rich than the first reformate exit stream 18) and a first liquid hydrocarbon exit stream 6 (less hydrogen-rich than the first reformate exit stream 18);
sending the first liquid hydrocarbon exit stream 6 to a first stabilization section 7 (optionally dedicated) so as to separate a first gaseous hydrocarbon compound exit stream 8 comprising C1-C2 hydrocarbon compounds (relative to the hydrocarbon exit stream), a first liquid stream 9 (or LPG for liquefied petroleum gas) comprising (predominantly, preferably essentially) C3-C4 or C3-C5 hydrocarbon compounds, and a first liquid fraction 10 (stabilized) comprising (predominantly, preferably essentially) hydrocarbon compounds having at least 4 or 5 carbon atoms and preferably at least 5 carbon atoms;
sending the first liquid fraction 10 to a first reformate separation column 11 (optionally dedicated) so as to separate a first C6-C8 fraction 12 comprising (predominantly, preferably essentially) C6-C8 hydrocarbon compounds and a first C8+ fraction 13 comprising (predominantly, preferably essentially) C8+ hydrocarbon compounds;
sending the first C6-C8 fraction 12 at least in part to the aromatics extraction unit 14.

According to one or more embodiments, the raffinate 16 is sent to a first dedicated hydrotreating unit 21 (for example for removal of sulfur, nitrogen, olefins) so as to produce a first hydrotreated exit stream 22 sent to the first catalytic reforming unit 17 (in place of the raffinate 16).

According to one or more embodiments, the first reformate separation column 11 is suitable for separating a first top fraction 25 comprising (predominantly, preferably essentially) C5 hydrocarbon compounds. According to one or more embodiments, the first reformate separation column 11 is suitable for sending the C5 hydrocarbon compounds to the aromatics extraction unit 14 with the first C6-C8 fraction 12.

According to one or more embodiments, the process further comprises sending the extract 15 and the first C8+ fraction 13 (preferably separately) to an aromatics production unit (aromatic complex), for example for producing xylenes (preferably para-xylene) and optionally benzene. According to one or more embodiments, the extract 15 is sent to a section for fractionating benzene and toluene of the aromatic complex, and the first C8+ fraction 13 is sent to a section for fractionating C8 and C9 aromatic compounds of the aromatic complex.

The term “recontacting section” is in the present description understood to mean a section comprising an operation that makes it possible to extract compounds present in a gaseous phase by means of a liquid phase that has an absorption capacity, by virtue of contact between the two phases. For example, recontacting can be achieved by effecting direct contact through inline mixing of the liquid and gaseous phases or in a recontacting device dedicated to the unit operation.

The term “predominantly” is in the present description understood to mean that the stream under consideration comprises at least 50% by weight of the components under consideration.

The term “essentially” is throughout the present text understood to mean that the stream under consideration comprises at least 80%, especially at least 90% or 95%, preferably at least 99% or 99.5%, by weight of said components. This may also be all of the components under consideration, apart from the usual impurities.

The term “dedicated” is in the present description understood to mean that the unit referred to as “dedicated” treats only the exit stream under consideration. For example, in the process according to the present invention, the first dedicated catalytic reforming unit 17 treats only the raffinate 16.

The term “stabilized” is in the present description understood to mean that the liquid fraction has been distilled to remove compounds having 4 carbon atoms or fewer (C4−), for example so as to contain essentially no C4− compounds.

The term “Cn hydrocarbon cut” is in the present description understood to mean a cut comprising hydrocarbons having n carbon atoms. Cn+ cut is understood to mean a cut comprising hydrocarbons having at least n carbon atoms. Cn− cut is understood to mean a cut comprising hydrocarbons having at most n carbon atoms.

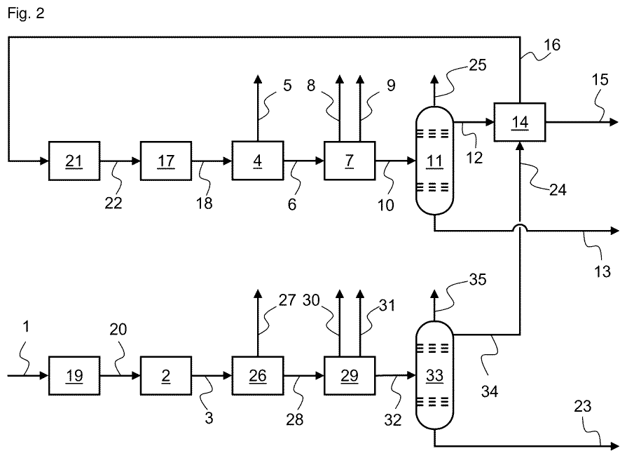

With reference to FIG. 2, according to one or more embodiments, the process comprises the following steps:
sending a second hydrocarbon feedstock 1 to a second catalytic reforming unit 2 (dedicated) so as to obtain a second reformate exit stream 3;
sending the second reformate exit stream 3 to a second recontacting section 26 (optionally dedicated) so as to obtain a second hydrogen-rich gaseous exit stream 27 (relative to the second reformate exit stream 3) and a second liquid hydrocarbon exit stream 28;
sending the second liquid hydrocarbon exit stream 28 to a second stabilization section 29 (optionally dedicated) so as to separate a second gaseous hydrocarbon compound exit stream 30 comprising C1-C2 hydrocarbon compounds (relative to the hydrocarbon exit stream), a second liquid stream 31 (or LPG for liquefied petroleum gas) comprising (predominantly, preferably essentially) C3-C4 or C3-C5 hydrocarbon compounds, and a second liquid fraction 32 (stabilized) comprising (predominantly, preferably essentially) hydrocarbon compounds having at least 4 or 5 carbon atoms and preferably at least 5 carbon atoms;
sending the second liquid fraction 32 to a second reformate separation column 33 (optionally dedicated) so as to separate a second C6-C8 fraction 34 and a second C8+ fraction 23 comprising (predominantly, preferably essentially) C8+ hydrocarbon compounds;
sending the second C6-C8 fraction 34 to the aromatics extraction unit 14 as said first C6-C8 hydrocarbon compound feedstock 24.

It is understood that the terms “first” and “second” used in the present description are employed simply to differentiate the different units and the different exit streams of the process according to the present invention. For example, when modifying (“revamping”) an existing production apparatus, the reformate separation column of the existing production apparatus can be considered to be the “first” reformate separation column (modification without adding an additional reformate separation column) or the “second” reformate separation column (modification with the addition of an additional reformate separation column). The same applies to the stabilization section (and optionally the recontacting section) of the existing production apparatus, which can be considered to be the “first” stabilization section (modification without adding an additional stabilization section) or “second” stabilization section (modification with the addition of an additional stabilization section).

According to one or more embodiments, the second hydrocarbon feedstock 1 is sent to a second dedicated hydrotreating unit 19 (dedicated) (for example for removal of sulfur, nitrogen, olefins) so as to produce a second hydrotreated exit stream 20 sent to the second catalytic reforming unit 2 (in place of the second hydrocarbon feedstock 1).

According to one or more embodiments, the second reformate separation column 33 is suitable for separating a second top fraction 35 comprising (predominantly, preferably essentially) C5 hydrocarbon compounds. According to one or more embodiments, the second reformate separation column 33 is suitable for sending the C5 hydrocarbon compounds to the aromatics extraction unit 14 with the second C6-C8 fraction 34.

According to one or more embodiments, the process further comprises sending the second C8+ fraction 23 at least in part to the aromatic complex. According to one or more embodiments, the second C8+ fraction 23 is sent to the section for fractionating C8 and C9 aromatic compounds of the aromatic complex, for example as a mixture with the first C8+ fraction 13.

According to the invention, the first C6-C8 hydrocarbon compound feedstock 24 comprises (consists of) aromatic compounds having 6 to 8 carbon atoms (for example benzene and toluene) and non-aromatic compounds having 6 to 8 carbon atoms (for example aliphatic compounds such as paraffinic and naphthenic compounds). According to one or more embodiments, the first C6-C8 hydrocarbon compound feedstock 24 comprises at least 50% by weight, preferably at least 60% by weight, very preferably at least 70% by weight, of aromatic compounds. According to one or more embodiments, the first C6-C8 hydrocarbon compound feedstock 24 comprises between 55% by weight and 95% by weight, preferably between 60% by weight and 90% by weight, for example between 65% by weight and 85% by weight, of aromatic compounds. According to one or more embodiments, the first C6-C8 hydrocarbon compound feedstock 24 comprises less than 50% by weight, preferably less than 60% by weight, very preferably less than 70% by weight, of non-aromatic compounds. According to one or more embodiments, the first C6-C8 hydrocarbon compound feedstock 24 comprises between 5% by weight and 45% by weight, preferably between 10% by weight and 40% by weight, for example between 15% by weight and 35% by weight, of non-aromatic compounds. According to one or more embodiments, the first C6-C8 hydrocarbon compound feedstock 24 comprises (predominantly, preferably essentially) C6-C7 hydrocarbon compounds and optionally C5 hydrocarbon compounds. According to one or more embodiments, the first C6-C8 hydrocarbon compound feedstock 24 is substantially free of compounds containing sulfur, nitrogen or oxygen.

According to one or more embodiments, the second hydrocarbon feedstock 1 comprises (consists of) naphtha, for example a naphtha having an initial distillation temperature of greater than or equal to 30° C. and a final distillation temperature of less than or equal to 220° C. (at atmospheric pressure). According to one or more embodiments, the second hydrocarbon feedstock 1 predominantly comprises C6 to C10 hydrocarbons and optionally C5 hydrocarbon compounds. According to one or more embodiments, the naphtha cut essentially comprises C6 to C10 hydrocarbons. According to one or more embodiments, the second hydrocarbon feedstock 1 predominantly comprises hydrocarbons in which the initial distillation temperature is between 70° C. and 90° C. or between 80° C. and 100° C. and in which the final distillation temperature is between 150° C. and 220° C. According to one or more embodiments, the second hydrocarbon feedstock 1 essentially comprises hydrocarbons in which the initial distillation temperature is between 80° C. and 100° C. and in which the final distillation temperature is between 150° C. and 220° C. According to one or more embodiments, the second hydrocarbon feedstock 1 comprises at least 40% of paraffinic and/or naphthenic compounds. According to one or more embodiments, the second hydrocarbon feedstock 1 comprises at least 80% of paraffinic and/or naphthenic compounds. According to one or more embodiments, the second hydrocarbon feedstock 1 comprises less than 20% of aromatic compounds.

According to one or more embodiments, the raffinate 16 predominantly comprises aliphatic compounds having 6 to 8 carbon atoms (for example non-aromatic compounds such as paraffinic and naphthenic compounds). According to one or more embodiments, the raffinate 16 comprises less than 70% by weight, preferably less than 80% by weight, very preferably less than 90% by weight, of aromatic compounds. According to one or more embodiments, the raffinate 16 essentially comprises non-aromatic compounds having from 6 to 8 carbon atoms. According to one or more embodiments, the raffinate 16 comprises (predominantly, preferably essentially) C6-C7 aliphatic compounds and optionally C5 aliphatic compounds.

A first, non-limiting example of the process according to the invention is given in FIG. 1, in which the first C6-C8 hydrocarbon compound feedstock 24 is sent to the aromatics extraction unit 14 so as to form the extract 15 (stream of aromatic compounds having 6 to 8 carbon atoms) and the raffinate 16 (stream of aliphatic compounds having 6 to 8 carbon atoms). The raffinate 16 is sent to the first optional hydrotreating unit 21 so as to produce the first hydrotreated exit stream 22, which is sent to the first catalytic reforming unit 17. The first reformate exit stream 18 from the first catalytic reforming unit 17 is sent to the first recontacting section 4 so as to form the first hydrogen-rich gaseous exit stream 5 and the first liquid hydrocarbon exit stream 6. The first liquid hydrocarbon exit stream 6 is sent to the first stabilization section 7 so as to recover the first gaseous hydrocarbon compound exit stream 8 enriched in C1 and C2 hydrocarbons, the first liquid stream 9 comprising liquefied petroleum gas (LPG), and also the first liquid fraction 10 predominantly containing hydrocarbons having at least 4 or 5 carbon atoms. The first liquid fraction 10 is sent to the first reformate separation column 11 so as to obtain the first C6-C8 fraction 12 comprising C6 to C8 compounds, the first C8+ fraction 13 (e.g. bottom stream) comprising C8+ aromatic compounds, and optionally the first top fraction 25 (C5 compounds). According to one or more embodiments, the first C6-C8 fraction 12 comprises (predominantly, preferably essentially) C6-C7 hydrocarbon compounds and optionally C5 hydrocarbon compounds. The first C6-C8 fraction 12 is sent to the aromatics extraction unit 14 and the first C8+ fraction 13 is sent to an aromatic complex.

A second non-limiting example of the process according to the invention is shown in FIG. 2, in which the second hydrocarbon feedstock 1 of naphtha type comprising C6 to C10 hydrocarbons is sent to the second optional hydrotreating unit 19 and then the second hydrotreated exit stream 20 is sent to the second catalytic reforming unit 2. The second reformate exit stream 3 from the second catalytic reforming unit 2 is sent to the second recontacting section 26 so as to form the second hydrogen-rich gaseous exit stream 27 and the second liquid hydrocarbon exit stream 28. The second liquid hydrocarbon exit stream 28 is sent to the second stabilization section 29 so as to recover the second gaseous hydrocarbon compound exit stream 30 enriched in C1 and C2 hydrocarbons, the second liquid stream 31 comprising liquefied petroleum gas (LPG), and the second liquid fraction 32 predominantly containing hydrocarbons having at least 4 or 5 carbon atoms. The second liquid fraction 32 is sent to the second reformate separation column 33 so as to obtain the second C6-C8 fraction 34 comprising C6 to C8 compounds and the second C8+ fraction 23 (e.g. bottom stream) comprising C8+ aromatic compounds. According to one or more embodiments, the second C6-C8 fraction 34 comprises (predominantly, preferably essentially) C6-C7 hydrocarbon compounds and optionally C5 hydrocarbon compounds. The second C6-C8 fraction 34 is sent to the aromatics extraction unit 14 in place of the first C6-C8 hydrocarbon compound feedstock 24.

According to one or more embodiments, the first catalytic reforming unit 17 and/or the second catalytic reforming unit 2 comprises at least one reforming reactor operated according to at least one of the following operating conditions:
a temperature of between 400° C. and 600° C., preferably between 470° C. and 570° C.;
a pressure of between 0.1 MPa and 3 MPa, preferably between 0.3 MPa and 2.5 MPa;
a molar ratio between hydrogen and hydrocarbon compounds of between 0.8 and 8 mole/mole; and
a mass flow rate of treated stream per unit mass of catalyst and per hour of between 0.1 h⁻¹ and 10 h⁻¹, preferably between 0.5 h⁻¹ and 6 h⁻¹, such as between 1 h⁻¹ and 5 h⁻¹.

According to one or more embodiments, the first catalyst used in the first catalytic reforming unit 17 comprises an active phase comprising at least one metal selected from platinum, zinc or molybdenum, and a support comprising a zeolite, and optionally a binder. Preferably, the metal is platinum.

According to one or more embodiments, the first catalyst contains an amount of metal of between 0.02% and 2% by weight, preferably between 0.05% and 1.5% by weight and more preferably between 0.1% and 0.8% by weight, relative to the total weight of the first catalyst.

According to one or more embodiments, the zeolite of the support of the first catalyst is selected from a zeolite L, a zeolite X, a zeolite Y or a zeolite ZSM-5. More preferably, the zeolite is a zeolite L.

According to one or more embodiments, the binder of the first catalyst is selected from aluminosilicates, alumina, silica, clays or silicon carbides, taken alone or in combination. More preferably, the binder is silica.

According to one or more embodiments, the first catalyst comprises an active phase comprising platinum, a support comprising a zeolite L, and optionally a binder of silica type.

According to one or more embodiments, the second catalyst used in the second catalytic reforming unit 2 comprises an active phase comprising at least one metal selected from nickel, ruthenium, rhodium, palladium, iridium or platinum, at least one promoter selected from rhenium, tin, germanium, cobalt, nickel, iridium, rhodium or ruthenium, and a support based on alumina, on silica/alumina or on silica. Preferably, the second catalyst comprises an active phase comprising platinum and tin.

According to one or more embodiments, the second catalyst contains an amount of the at least one metal (for example platinum) of between 0.02% and 2% by weight, preferably between 0.05% and 1.5% by weight and more preferably between 0.1% and 0.8% by weight, relative to the total weight of the second catalyst.

According to one or more embodiments, the support of the second catalyst is based on alumina. According to one or more embodiments, at least one alumina of the porous support used in the second catalyst is of the chi, eta, gamma or delta type. Preferably, the at least one alumina of the porous support is of the gamma or delta type. More preferably, the at least one alumina of the porous support is of the gamma type.

The second catalyst may also comprise at least one dopant metal selected from the group formed by gallium, gold, nickel, rhenium, barium, silver, iron, bismuth, indium, yttrium and the lanthanides (cerium, dysprosium, ytterbium), taken alone or as a mixture. The content of each dopant metal relative to the total weight of the second catalyst is between 0% and 2% by weight, preferably 0.01% and 1% by weight, preferably between 0.01% and 0.7% by weight, relative to the total weight of the second catalyst.

The second catalyst may also comprise at least one halogen used to acidify the alumina support. The halogen content may represent between 0.1% and 15% by weight relative to the total weight of the second catalyst, preferably 0.2% and 5% by weight relative to the total weight of the second catalyst. Preference is given to using just one halogen, in particular chlorine or fluorine. When the second catalyst comprises just one halogen that is chlorine or fluorine, the chlorine content is between 0.5% and 2% by weight relative to the total weight of the second catalyst.

The second catalyst may also comprise an alkali metal in proportions of the order of 0.1% to 3% by weight relative to the total weight of the second catalyst. Preferably, the alkali metal is potassium.

According to one or more embodiments, the second catalyst comprises an active phase comprising platinum and tin, a promoter comprising tin, and a support based on γ-type alumina.

According to one or more embodiments, the second hydrocarbon feedstock 1 and/or the raffinate 16 undergoes a dedicated hydrotreating step (for example desulfurization) before the catalytic reforming steps.

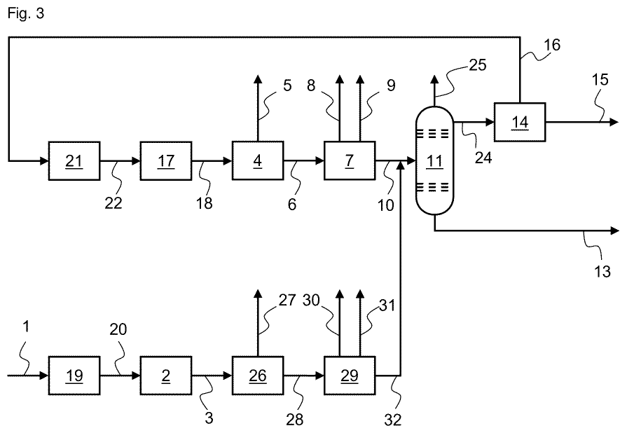

In the example in FIG. 2, the first reformate separation column 11 and the second reformate separation column 33 are different columns, the first being dedicated to separating the first liquid fraction 10 and the second being dedicated to separating the second liquid fraction 32, respectively. With reference to FIG. 3, according to one or more embodiments, the first liquid fraction 10 and the second liquid fraction 32 are sent to the first reformate separation column 11. In this/these embodiment(s), the second reformate separation column 33 is absent/not used. Accordingly, the first C8+ fraction 13 comprises the second C8+ fraction 23, the first top fraction 25 comprises the second top fraction 35, and the first C6-C8 hydrocarbon compound feedstock 24 comprises the first C6-C8 fraction 12.

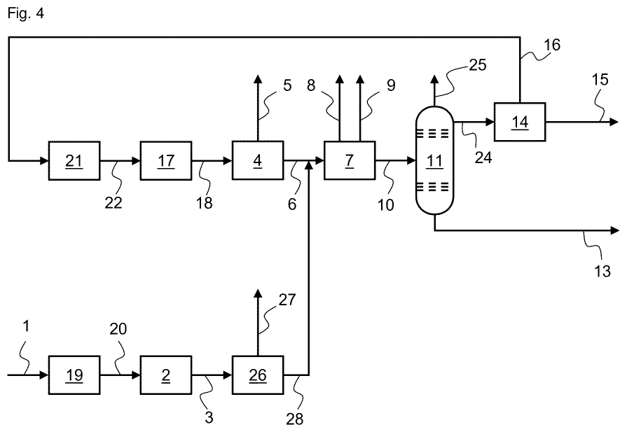

In the example in FIG. 3, the first stabilization section 7 and the second stabilization section 29 are different sections, the first being dedicated to stabilizing the first liquid hydrocarbon exit stream 6 and the second being dedicated to stabilizing the second liquid hydrocarbon exit stream 28, respectively. With reference to FIG. 4, according to one or more embodiments, the first liquid hydrocarbon exit stream 6 and the second liquid hydrocarbon exit stream 28 are sent to the first stabilization section 7. In this/these embodiment(s), the second stabilization section 29 is absent/not used. Accordingly, the first gaseous hydrocarbon compound exit stream 8 comprises the second gaseous hydrocarbon compound exit stream 30, the first liquid stream 9 comprises the second liquid stream 31, and the first liquid fraction 10 comprises the second liquid fraction 32.

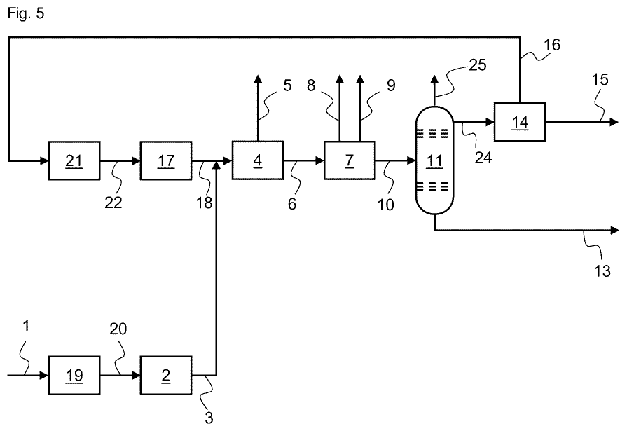

In the example in FIG. 4, the first recontacting section 4 and the second recontacting section 26 are different sections, the first being dedicated to recontacting the first reformate exit stream 18 and the second being dedicated to recontacting the second reformate exit stream 3, respectively. With reference to FIG. 5, according to one or more embodiments, the first reformate exit stream 18 and the second reformate exit stream 3 are sent to the first recontacting section 4. In this/these embodiment(s), the second recontacting section 26 is absent/not used. Accordingly, the first hydrogen-rich gaseous exit stream 5 comprises the second hydrogen-rich gaseous exit stream 27 and the first liquid hydrocarbon exit stream 6 comprises the second liquid hydrocarbon exit stream 28.

According to one or more embodiments, the recontacting and stabilizing steps (sections 4, 7, 26 and 29) of the process comprise the following substeps:
i) separating the reformate stream (for example the first reformate exit stream 18) into a gaseous phase and a liquid phase containing hydrocarbons;
ii) cooling the liquid phase from step i) to a temperature less than or equal to 45° C., preferably less than 25° C., very preferably less than 15° C., by means of a cooling device;
iii) effecting a first recontacting of the cooled liquid phase with the gaseous phase in a separation means so as to recover a hydrogen-rich gaseous exit stream (for example the first hydrogen-rich gaseous exit stream 5) and a liquid hydrocarbon exit stream (for example the first liquid hydrocarbon exit stream 6);
iv) effecting a second recontacting of the liquid hydrocarbon exit stream with a recycle gas and separating a gaseous exit stream rich in hydrocarbon compounds (for example the first gaseous hydrocarbon compound exit stream 8) and a second liquid hydrocarbon exit stream;
v) fractionating the second liquid hydrocarbon exit stream from step iv) in a fractionating column so as to separate a gaseous fraction and a liquid fraction (for example the first liquid fraction 10);
vi) condensing the gaseous fraction from step v) and separating a liquid stream (for example the first liquid stream 9) and the recycle gas, which is recycled to step iv). Details of these recontacting and stabilization steps are set out in patent FR3074175B1.

According to one or more embodiments, the separation means is a recontacting column operating countercurrently or a separating drum.

Advantageously, steps i) to vi) use the frigories contained in the gaseous or liquid exit streams generated in the recontacting step carried out in a recontacting (or absorption) column to precool the liquid hydrocarbon phase before the latter undergoes a cooling that enables it to reach the desired temperature for the recontacting step. The thermal integration thus makes it possible to significantly reduce cold utility consumption and therefore the overall energy consumption of the process.

According to one or more embodiments, before the cooling step ii), the liquid phase from step i) is precooled by heat exchange in an exchanger fed with the hydrogen-rich gaseous exit stream and the liquid hydrocarbon exit stream from step iii).

According to one or more embodiments, before the cooling step ii), the liquid phase from step i) undergoes heat exchange in an exchanger fed with the hydrogen-rich gaseous exit stream, and the gaseous phase from step i) undergoes heat exchange in an exchanger fed with the liquid hydrocarbon exit stream.

According to one or more embodiments, before the cooling step ii), the liquid phase from step i) undergoes heat exchange in an exchanger fed with the liquid hydrocarbon exit stream, and the gaseous phase from step i) undergoes heat exchange in an exchanger fed with the hydrogen-rich gaseous exit stream.

According to one or more embodiments, part or all of the gaseous exit stream rich in hydrocarbon compounds is recycled before the first recontacting step.

According to one or more embodiments, the gaseous exit stream rich in hydrocarbon compounds is recycled as a mixture with the gaseous phase from step i).

### EXAMPLES

### Example 1: Conventional Reforming

Example 1 is noninventive and corresponds to a conventional reforming scheme without upgrading the raffinate 16 from the extraction of aromatics.

Relative to FIG. 2, example 1 does not include the following units:
first dedicated hydrotreating unit 21;
first dedicated catalytic reforming unit 17;
first recontacting section 4;
first stabilization section 7;
first reformate separation column 11.

The total input flow is 187.2 t/h. 126 t/h is converted into aromatics. 54.2% by weight of the second hydrocarbon feedstock 1 is converted into para-xylene and 13.1% by weight into benzene after treatment of the extract 15 and of the second C8+ fraction 23 in an aromatic complex for the production of para-xylene and benzene. The input and output flow rates of the process in example 1 are listed in Table 1 below.

TABLE 1

| | t/h |
|---|---|
| Catalytic reforming (noninventive) | |
| Second hydrocarbon feedstock 1 | 187.2 |
| Second hydrogen-rich gaseous exit stream 27 | 10.9 |
| Second gaseous hydrocarbon compound exit stream 30 | 19.5 |
| Second liquid stream 31 | 4.4 |
| Second top fraction 35 | 4.8 |
| Raffinate 16 (not recycled) | 15.8 |
| Extract 15 | 57.5 |
| Second C8+ fraction 23 | 74.3 |
| Aromatic complex (downstream process | |
| known to those skilled in the art) | |
| Benzene | 24.6 |
| Para-xylene | 101.4 |
| Heavier | 5.8 |

### Example 2: Separation of the Reformer Input Feedstock

Example 2 is noninventive and corresponds to a reforming scheme with separation of the second hydrocarbon feedstock 1 for the parallel reforming of a light fraction on the one hand and of a heavy fraction on the other (see FIG. 1 of FR3074175B1).

Relative to FIG. 2, example 2 does not include the following units:
first dedicated hydrotreating unit 21;
first dedicated catalytic reforming unit 17;
first recontacting section 4;
first stabilization section 7;
first reformate separation column 11.

Relative to FIG. 2, the second hydrotreating unit 19 and the second catalytic reforming unit 2 of example 2 are each divided into two units so as to treat the light fraction on the one hand and the heavy fraction on the other.

The total input flow is 187.2 t/h. 140.6 t/h is converted into aromatics. 56.4% by weight of the second hydrocarbon feedstock 1 is converted into para-xylene and 18.7% by weight into benzene after treatment of the extract 15 and of the second C8+ fraction 23 in an aromatic complex for the production of para-xylene and benzene. The input and output flow rates of the process in example 2 are listed in Table 2 below.

TABLE 2

| | t/h |
|---|---|
| Catalytic reforming (noninventive) | |
| Second hydrocarbon feedstock 1 | 187.2 |
| Second hydrogen-rich gaseous exit stream 27 | 13.4 |
| Second gaseous hydrocarbon compound exit stream 30 | 19.4 |
| Second liquid stream 31 | 2.6 |
| Second top fraction 35 | 4.6 |
| Raffinate 16 | 0 |
| Extract 15 | 70.4 |
| Second C8+ fraction 23 | 76.8 |
| Aromatic complex (downstream process | |
| known to those skilled in the art) | |
| Benzene | 35.1 |
| Para-xylene | 105.5 |
| Heavier | 6.6 |

### Example 3: Dedicated Raffinate Reforming

Example 3 is inventive and corresponds to a scheme described in FIG. 2.

The 15.8 t/h of raffinate 16 not upgraded in example 1 is sent to the first catalytic reforming unit 17. This makes it possible to increase the production of benzene and para-xylene from 126 t/h to 137.8 t/h, an increase of about 10% in total aromatics upgraded. The yield of upgraded aromatics goes from 67.3% by weight to 73.6% by weight. 57.8% by weight of the second hydrocarbon feedstock 1 is converted into para-xylene and 15.9% by weight into benzene.

Relative to example 2, slightly less benzene and para-xylene are produced (137.8 t/h vs. 140.6 t/h). On the other hand, it is advantageous that more para-xylene is produced (108 t/h vs. 105 5 t/h), which is more valuable than benzene. The selectivity for para-xylene relative to benzene is better in example 3 than in example 2 (78.4% by weight vs. 75.0% by weight of para-xylene). The input and output flow rates of the process in example 3 are listed in Table 3 below.

TABLE 3

| | t/h |
|---|---|
| Catalytic reforming (inventive) | |
| Second hydrocarbon feedstock 1 | 187.2 |
| First hydrogen-rich gaseous exit stream 5 | 0.9 |
| First gaseous hydrocarbon compound exit stream 8 | 1.6 |
| First liquid stream 9 | 0.3 |
| First top fraction 25 | 0.4 |
| Second hydrogen-rich gaseous exit stream 27 | 10.9 |
| Second gaseous hydrocarbon compound exit stream 30 | 19.5 |
| Second liquid stream 31 | 4.4 |
| Second top fraction 35 | 4.8 |
| Raffinate 16 (recycled to first dedicated hydrotreating unit 21) | 15.8 |
| Extract 15 | 68.3 |
| First C8+ fraction 13 | 5.9 |
| Second C8+ fraction 23 | 70.2 |
| Aromatic complex (downstream process | |
| known to those skilled in the art) | |
| Benzene | 29.8 |
| Para-xylene | 108.0 |
| Heavier | 6.6 |

1. Process for producing aromatic compounds from a hydrocarbon compound feedstock, comprising the following steps:
sending a first C6-C8 hydrocarbon compound feedstock (24) to an aromatics extraction unit (14) so as to produce an extract (15) comprising aromatic compounds having 6 to 8 carbon atoms and a raffinate (16) comprising aliphatic compounds having 6 to 8 carbon atoms;
sending the raffinate (16) to a first dedicated catalytic reforming unit (17) so as to produce a first reformate exit stream (18);
sending the first reformate exit stream (18) to a first recontacting section (4) so as to separate a first hydrogen-rich gaseous exit stream (5) and a first liquid hydrocarbon exit stream (6);
sending the first liquid hydrocarbon exit stream (6) to a first stabilization section (7) so as to separate a first gaseous hydrocarbon compound exit stream (8) comprising C1-C2 hydrocarbon compounds, a first liquid stream (9) comprising C3-C4 or C3-C5 hydrocarbon compounds, and a first liquid fraction (10) comprising hydrocarbon compounds having at least 4 or 5 carbon atoms and preferably at least 5 carbon atoms;
sending the first liquid fraction (10) to a first reformate separation column (11) so as to separate a first C6-C8 fraction (12) comprising C6-C8 hydrocarbon compounds and a first C8+ fraction (13) comprising hydrocarbon compounds having at least 8 carbon atoms;
sending the first C6-C8 fraction (12) at least in part to the aromatics extraction unit (14).

2. Process according to claim 1, comprising sending the extract (15) and the first C8+ fraction (13) to an aromatics production unit.

3. Process according to claim 1, wherein the raffinate (16) is sent to a first dedicated hydrotreating unit (21) so as to produce a first hydrotreated exit stream (22) sent to the first catalytic reforming unit (17).

4. Process according to claim 1, wherein the first C6-C8 fraction (12) comprises C5 hydrocarbon compounds, and/or a first top fraction (25) comprising C5 hydrocarbon compounds is separated in the first reformate separation column (11).

5. Process according to claim 1, wherein the first C6-C8 hydrocarbon compound feedstock (24) comprises at least 50% by weight, preferably at least 60% by weight, very preferably at least 70% by weight, of aromatic compounds.

6. Process according to claim 1, wherein the first catalytic reforming unit (17) comprises at least one reforming reactor operated according to at least one of the following operating conditions:
a temperature of between 400° C. and 600° C., preferably between 470° C. and 570° C.;
a pressure of between 0.1 MPa and 3 MPa, preferably between 0.3 MPa and 2.5 MPa;
a molar ratio between hydrogen and hydrocarbon compounds of between 0.8 and 8 mole/mole; and
a mass flow rate of treated stream per unit mass of catalyst and per hour of between 0.1 h⁻¹ and 10 h⁻¹, preferably between 0.5 h⁻¹ and 6 h⁻¹;
optional catalyst comprising: an active phase comprising at least one metal selected from platinum, zinc or molybdenum, preferably platinum; a support comprising a zeolite, preferably a zeolite selected from a zeolite L, a zeolite X, a zeolite Y or a zeolite ZSM-5; and optionally a binder selected from aluminosilicate, alumina, silica, clays or silicon carbides, taken alone or in combination.

7. Process according to claim 1, comprising the following steps:
sending a second hydrocarbon feedstock (1) to a second catalytic reforming unit (2) so as to produce a second reformate exit stream (3);
sending the second reformate exit stream (3) to a second recontacting section (26) so as to produce a second hydrogen-rich gaseous exit stream (27) and a second liquid hydrocarbon exit stream (28);
sending the second liquid hydrocarbon exit stream (28) to a second stabilization section (29) so as to separate a second gaseous hydrocarbon compound exit stream (30) comprising C1-C2 hydrocarbon compounds, a second liquid stream (31) comprising C3-C4 or C3-C5 hydrocarbon compounds, and a second liquid fraction (32) comprising hydrocarbon compounds having at least 4 or 5 carbon atoms and preferably at least 5 carbon atoms;
sending the second liquid fraction (32) to a second reformate separation column (33) so as to separate a second C6-C8 fraction (34) and a second C8+ fraction (23) comprising hydrocarbon compounds having at least 8 carbon atoms;
sending the second C6-C8 fraction (34) at least in part to the aromatics extraction unit (14) as said first C6-C8 hydrocarbon compound feedstock (24).

8. Process according to claim 7, comprising sending the second C8+ fraction (23) at least in part to an aromatic complex.

9. Process according to claim 1, comprising the following steps:
sending a second hydrocarbon feedstock (1) to a second catalytic reforming unit (2) so as to produce a second reformate exit stream (3);
sending the second reformate exit stream (3) to a second recontacting section (26) so as to produce a second hydrogen-rich gaseous exit stream (27) and a second liquid hydrocarbon exit stream (28);
sending the second liquid hydrocarbon exit stream (28) to a second stabilization section (29) so as to separate a second gaseous hydrocarbon compound exit stream (30) comprising C1-C2 hydrocarbon compounds, a second liquid stream (31) comprising C3-C4 or C3-C5 hydrocarbon compounds, and a second liquid fraction (32) comprising hydrocarbon compounds having at least 4 or 5 carbon atoms and preferably at least 5 carbon atoms;
sending the second liquid fraction (32) to the first reformate separation column (11).

10. Process according to claim 1, comprising the following steps:
sending a second hydrocarbon feedstock (1) to a second catalytic reforming unit (2) so as to produce a second reformate exit stream (3);
sending the second reformate exit stream (3) to a second recontacting section (26) so as to produce a second hydrogen-rich gaseous exit stream (27) and a second liquid hydrocarbon exit stream (28);
sending the second liquid hydrocarbon exit stream (28) to the first stabilization section (7).

11. Process according to claim 1, comprising the following steps:
sending a second hydrocarbon feedstock (1) to a second catalytic reforming unit (2) so as to produce a second reformate exit stream (3);
sending the second reformate exit stream (3) to the first recontacting section (4).

12. Process according to claim 7, wherein the second hydrocarbon feedstock (1) is sent to a second dedicated hydrotreating unit (19) so as to produce a second hydrotreated exit stream (20) sent to the second catalytic reforming unit (2).

13. Process according to claim 7, wherein the second hydrocarbon feedstock (1) comprises naphtha.

14. Process for modifying an apparatus for producing aromatic compounds from a hydrocarbon compound feedstock,
the apparatus for producing aromatic compounds comprising:
a second catalytic reforming unit (2) suitable for treating a second hydrocarbon feedstock (1) and producing a second reformate exit stream (3);
a second recontacting section (26) suitable for treating the second reformate exit stream (3) and producing a second hydrogen-rich gaseous exit stream (27) and a second liquid hydrocarbon exit stream (28);
a second stabilization section (29) suitable for treating the second liquid hydrocarbon exit stream (28) and separating a second gaseous hydrocarbon compound exit stream (30) comprising C1-C2 hydrocarbon compounds, a second liquid stream (31) comprising C3-C4 or C3-C5 hydrocarbon compounds, and a second liquid fraction (32) comprising hydrocarbon compounds having at least 4 or 5 carbon atoms and preferably at least 5 carbon atoms;
a second reformate separation column (33) suitable for treating the second liquid fraction (32) and separating a second C6-C8 fraction (34) and a second C8+ fraction (23) comprising hydrocarbon compounds having at least 8 carbon atoms;
an aromatics extraction unit (14) suitable for treating the second C6-C8 fraction (34) and producing an extract (15) comprising aromatic compounds having 6 to 8 carbon atoms and a raffinate (16) comprising aliphatic compounds having 6 to 8 carbon atoms, the process comprising the following steps:
installing in the apparatus for producing aromatic compounds a first dedicated catalytic reforming unit (17) suitable for treating the raffinate (16), producing a first reformate exit stream (18), and sending the first reformate exit stream (18) to the second recontacting section (26) or to a first dedicated recontacting section (4);
optionally, installing in the apparatus for producing aromatic compounds a first dedicated recontacting section (4) suitable for treating the first reformate exit stream (18), separating a first hydrogen-rich gaseous exit stream (5) and a first liquid hydrocarbon exit stream (6), and sending the first liquid hydrocarbon exit stream (6) to the second stabilization section (29) or to a first dedicated stabilization section (7);
optionally, installing in the apparatus for producing aromatic compounds a first dedicated stabilization section (7) suitable for treating the first liquid hydrocarbon exit stream (6), separating a first gaseous hydrocarbon compound exit stream (8) comprising C1-C2 hydrocarbon compounds, a first liquid stream (9) comprising C3-C4 or C3-C5 hydrocarbon compounds, and a first liquid fraction (10) comprising hydrocarbon compounds having at least 4 or 5 carbon atoms and preferably at least 5 carbon atoms, and sending the first liquid fraction (10) to the second reformate separation column (33) or to a first dedicated reformate separation column (11);
optionally, installing in the apparatus for producing aromatic compounds a first dedicated reformate separation column (11) suitable for treating the first liquid fraction (10), separating a first C6-C8 fraction (12) comprising C6-C8 hydrocarbon compounds and a first C8+ fraction (13) comprising hydrocarbon compounds having at least 8 carbon atoms, and sending the first C6-C8 fraction (12) at least in part to the aromatics extraction unit (14).

15. Apparatus for producing aromatic compounds from a hydrocarbon compound feedstock, comprising the following elements:
an aromatics extraction unit (14) suitable for treating a first C6-C8 hydrocarbon compound feedstock (24) so as to produce an extract (15) comprising aromatic compounds having 6 to 8 carbon atoms and a raffinate (16) comprising aliphatic compounds having 6 to 8 carbon atoms;
a first dedicated catalytic reforming unit (17) suitable for treating the raffinate (16) and producing a first reformate exit stream (18);
a first recontacting section (4) suitable for treating the first reformate exit stream (18) and separating a first hydrogen-rich gaseous exit stream (5) and a first liquid hydrocarbon exit stream (6);
a first stabilization section (7) suitable for treating the first liquid hydrocarbon exit stream (6) and separating a first gaseous hydrocarbon compound exit stream (8) comprising C1-C2 hydrocarbon compounds, a first liquid stream (9) comprising C3-C4 or C3-C5 hydrocarbon compounds, and a first liquid fraction (10) comprising hydrocarbon compounds having at least 4 or 5 carbon atoms and preferably at least 5 carbon atoms;
a first reformate separation column (11) suitable for treating the first liquid fraction (10), separating a first C6-C8 fraction (12) comprising C6-C8 hydrocarbon compounds and a first C8+ fraction (13) comprising hydrocarbon compounds having at least 8 carbon atoms, and sending the first C6-C8 fraction (12) at least in part to the aromatics extraction unit (14).
